(19) United States
(12) Patent Application Publication
(10) Pub. No.: US 2013/0197399 A1
(43) Pub. Date: Aug. 1, 2013

(54) APPARATUSES AND METHODS FOR EVALUATING A PATIENT

(71) Applicant: Erwin B. Montgomery, Birmingham, AL (US)

(72) Inventor: Erwin B. Montgomery, Birmingham, AL (US)

(21) Appl. No.: 13/814,396

(22) PCT Filed: Jul. 21, 2011
(86) PCT No.: PCT/US11/44828

§ 371 Date: Feb. 20, 2013

Related U.S. Application Data
(60) Provisional application No. 61/370,997, filed on Aug. 5, 2010.

(51) Int. Cl. A61B 5/11; A61B 5/00; A61B 5/22

(52) U.S. Cl. CPC A61B 5/1107; A61B 5/224; A61B 5/6806
USPC 600/595

(57) ABSTRACT

In one embodiment, a patient evaluation apparatus includes a glove body adapted to be worn on an examiner's hand, finger orientation sensors mounted to the glove body adapted to sense the orientation of the fingers and thumb of the examiner's hand, force sensors mounted to the glove body adapted to measure forces applied against the examiner's hand, and a motion sensor mounted to the glove body adapted to detect motion of the examiner's hand.

This application claims priority to copending U.S. provisional application entitled, “Appartuses And Methods For Measuring Muscular Force Generation And Resistance To Passive Movement,” having Ser. No. 61/370,997, filed Aug. 5, 2010, which is entirely incorporated herein by reference.

### BACKGROUND

It is sometimes necessary to gauge a patient's muscle strength or muscle tone. In such circumstances, an examiner typically makes determinations as to those characteristics by manually interfacing with the patient. For example, to gauge a patient's upper arm strength, the examiner may grip the patient's forearm and provide resistance to the patient's attempted flexion or extension of the arm to observe the amount of force with which the patient moves his or her arm. To gauge the muscle tone in the arm, the examiner may instead move the patient's relaxed arm and observe the passive resistance to such movement.

Although such techniques provide a general idea as to the patient's muscle strength or muscle tone, the conclusions that are drawn from the evaluation are completely subjective. Therefore, different examiners may reach different conclusions as to the condition of the patient under similar circumstances. In view of this subjectivity, various machines have been developed that can objectively measure muscle strength and muscle tone. Such machines either provide resistance to patient movement or move the relaxed patient in similar manner to how a human examiner would, all while measuring the actual forces at work. Although such machines provide the benefit of objective measurement, they typically are specifically designed for use with a particular body part and therefore are limited in their application. Furthermore, such machines tend to be cumbersome and expensive, thereby limiting their practical use.

In view of the above discussion, it can be appreciated that it would be desirable to have a means for evaluating muscle strength or muscle tone that does not suffer from one or more of the aforementioned drawbacks.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a top view of an embodiment of a patient evaluation apparatus.

FIG. 2 is a bottom view of the patient evaluation apparatus of FIG. 1.

FIG. 3 is side view of the patient evaluation apparatus of FIGS. 1 and 2 worn by a user who is gripping an object.

FIGS. 4A and 4B illustrate a further embodiment of a patient evaluation apparatus being used to evaluate a patient.

### DETAILED DESCRIPTION

As described above, evaluation of a patient's muscle strength or muscle tone can be too subjective when the examiner (e.g., physician) manually interfaces with the patient. Furthermore, machines designed to objectively measure these parameters tend to be limited in application, cumbersome, and expensive. Disclosed herein, however, are apparatuses for evaluating muscle strength and/or muscle tone that can be worn by the examiner while he or she manually interfaces with the patient. Therefore, the disclosed apparatuses combine the flexibility of manual evaluation with the objective measurement that comes from using a machine. In one embodiment, a patient evaluation apparatus comprises a device that is worn on the examiner's hand like a glove. The device is provided with instruments that can measure various parameters that are relevant to assessing muscle strength and/or muscle tone in patients, such as patients with neurological and/or orthopedic disorders.

In the following disclosure, various embodiments are described. It is to be understood that those embodiments are example implementations of the disclosed inventions and that alternative embodiments are possible. All such embodiments are intended to fall within the scope of this disclosure.

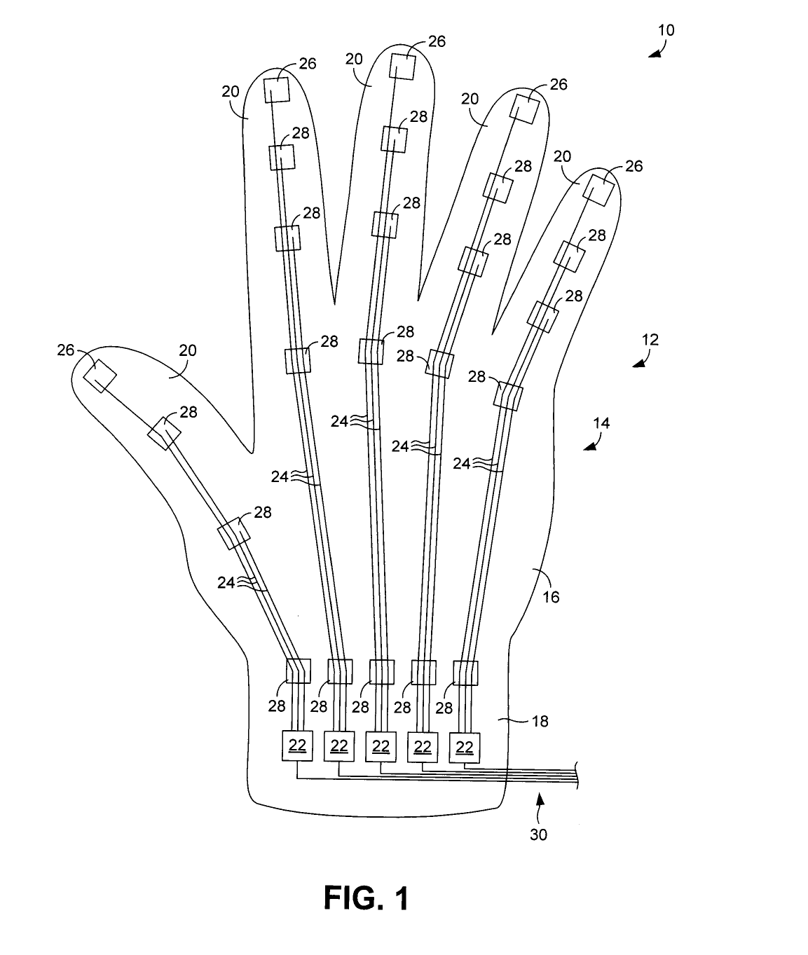

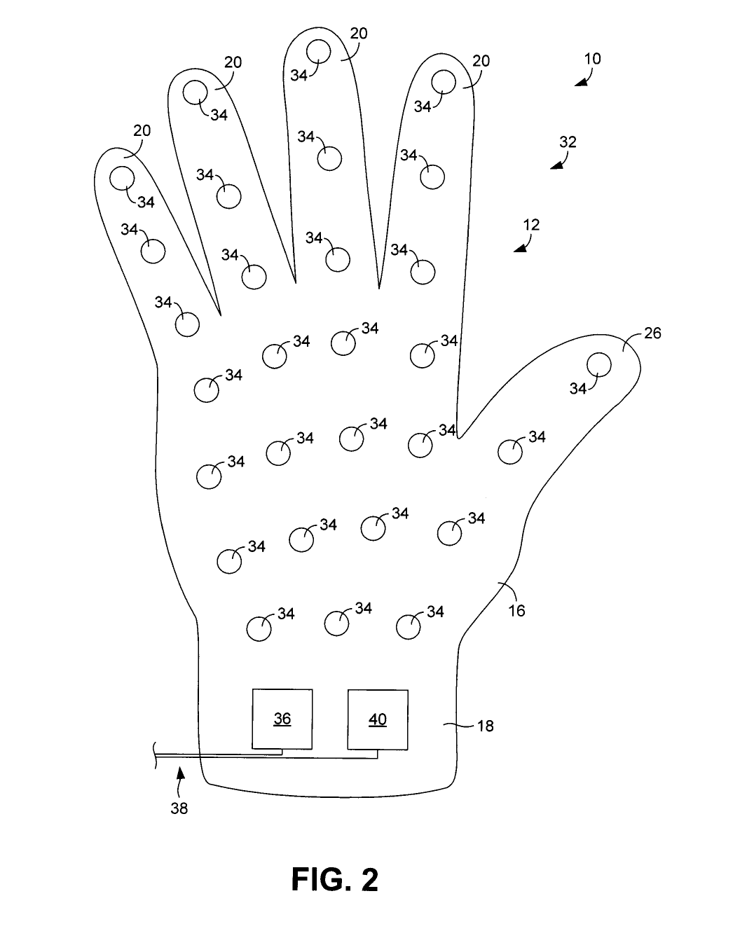

FIGS. 1 and 2 illustrate an embodiment of a patient evaluation apparatus 10 that can be worn on an examiner's hand like a glove. The apparatus 10 can therefore be described as a glove or glove-like device. For the remainder of the disclosure, the apparatus 10 will be generally referred to as a glove device 10.

FIG. 1 shows a top or outer side 12 of the glove device 10, which is adapted to cover the top or outer side of an examiner's hand. As is apparent from FIG. 1, the glove device 10 generally comprises a glove body 14 that, in the illustrated embodiment, has the shape, size, and configuration of a typical glove. Therefore, the body 14 includes a central portion 16 adapted to fit or wrap around the central portion of the hand, a cuff portion 18 adapted to fit or wrap around the wrist, and multiple finger sleeves 20 that are adapted to individually fit or wrap around the fingers and thumb. By way of example, the body 14 is made of a flexible cloth or mesh material that easily conforms to the contours of the examiner's hand. In some embodiments, the cloth or mesh is made of an elastic material. The body 14 can comprise a single layer of material, or multiple layers of material in which case various instruments (discussed below) can be mounted between adjacent layers of the body.

Although a conventional glove-like configuration that is adapted to fully wrap around and enclose the hand and fingers is shown in FIGS. 1 and 2, it is noted that such a configuration is not required. Instead, the glove body 14 need only support the various instruments the device 10 includes. Therefore, the body 14 could, for example, comprise multiple strips of material that wrap around the fingers and hand that support the device's instruments.

As is further illustrated in FIG. 1, the glove device 10 includes multiple finger orientation sensors that detect and measure the extent to which each finger is bent (i.e., flexed). More specifically, the finger orientation sensors can be used to determine the joint angle of each inter-phalangeal and phalangeal-metacarpal joint. As described below, determination of those joint angles enables calculation of a vector that identifies the magnitude and direction of a net force imposed upon the glove device 10 and the examiner's hand.

In one embodiment, the finger orientation sensors comprise linear potentiometers 22 that are mounted on or within the glove body 14 of the device 10 (e.g., at or near the cuff portion 18) and strands 24, such as strings or cables, that extend from the potentiometers to discrete locations of the body. In the illustrated embodiment, there are three strands 24 associated with each finger sleeve 20, and therefore each finger or thumb of the examiner. By way of example, a proximal end of each stand 24 is connected to a linear potentiometer 22 and a distal end of each strand is attached to the body 14 near the either the tip of the finger sleeve 20 or near a position on the finger sleeve that corresponds to a joint of the examiner. In such a case, the joint angle for each finger or thumb joint can be individually determined from the extent of extension of the strands 24 from the potentiometers 22, which correlate such extension with finger flexion.

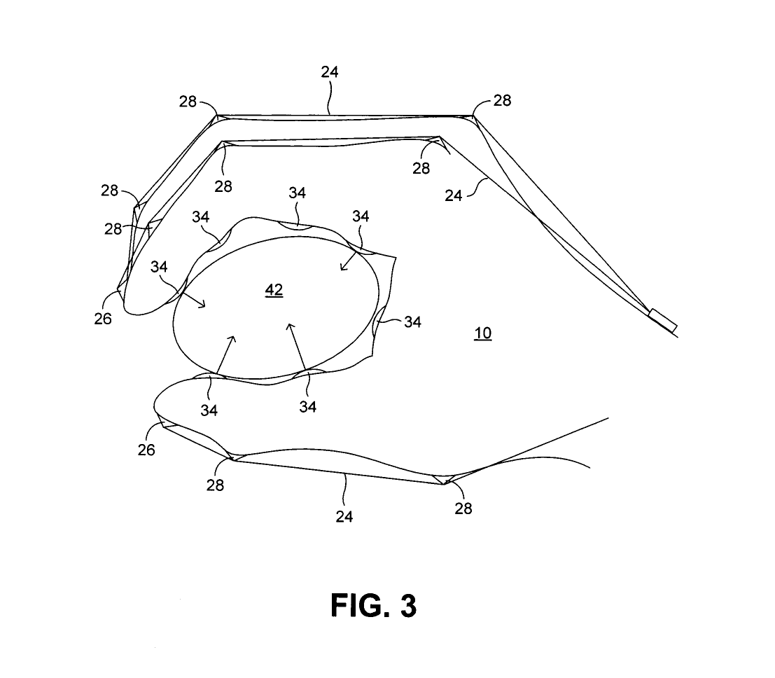

In some embodiments, the strands 24 attach to the tips of the finger sleeves 20 with mounting elements 26 that are provided on or within the glove body 14. As is shown in FIG. 1, the glove device 10 can further include guides 28 that guide the strands 24 from their linear potentiometers 22 to the mounting elements 26. Although the guides 28 limit lateral displacement of the strands 24, the strands are free to linearly slide relative to the guides so that the guides enable linear displacement of the strands. In some embodiments, the guides 28 comprise discrete fulcrum elements (see FIG. 3) that extend outward from the body 14. As is also shown in FIG. 1, the strands 24 that do not extend to the tips of the finger sleeves 20 can, optionally, attached at their distal ends to the guides 28. Although discrete fulcrum elements are shown in the figures, it is note that the guides can comprise any components that restrict lateral movement but enable linear movement of the strands 24. For example, the guides can alternatively comprise tubes or channels through which the strands 24 extend.

In alternative embodiments, the finger orientation sensors can be optical fiber-based devices that correlate bending loss to finger or thumb flexion. In such embodiments, each strand 24 comprises an optical fiber that extends between a first mounting element at 22 and a second mounting element at 26. Light can travel along the core of the optical fibers from the first mounting element 22 and can be reflected back by the second mounting element 26. When the fingers or thumbs are flexed, the optical fibers bend and some of the light will escape the core as the result of bending loss. The degree to which light escapes provides an indication of the degree of finger or thumb flexion. In some embodiments, the first mounting elements 22 comprise light sources (e.g., light emitting diodes) and light sensors (e.g., photodiodes) that respectively generate and sense light, and the second mounting elements 26 comprise reflecting elements (e.g., mirrors) that reflect light back along the length of the fiber. In other embodiments, the light sources and light sensors are comprised by a separate device to which the glove device 10 is connected.

Also shown in FIG. 1 are several lines 30 that extend from the linear potentiometers (or optical fiber mounting elements) 22. The lines 30 can be bundled within a cable that extends from the glove device 10 to another device, such as a computer and/or a light source and sensor. In some embodiments, the lines comprise one or more of data lines, power lines, and further optical fibers.

FIG. 2 illustrates a bottom or inner side 32 of the glove device 10, which is adapted to cover the bottom or inner side (palm) of the examiner's hand. As is shown in FIG. 2, multiple force sensors 34 are provided on the glove body 14 that are adapted to detect and measure the forces imparted to the examiner's hand by an object, such as a limb of a patient. In some embodiments, multiple force sensors 34 are provided on each finger sleeve 20 in positions that each corresponds to a pad of the examiner's finger or thumb. In addition multiple force sensors 34 are provided on the body 14 at positions that correspond to the palm of the examiner's hand. By way of example, the force sensors 34 can comprise electrical sensors, such as pressure transducers, piezoelectric sensors, or strain gauges. In other embodiments, the force sensors 34 can be hydraulic or pneumatic pads filled with a fluid.

As is further shown in FIG. 2, the glove device 10 can further comprise a component 36 that is in communication with each of the force sensors 34, and in some cases the potentiometers 22. In some embodiments, the component 36 comprises a digital signal processor that receives signals from the force sensors 34 and forwards them on to another device, such as a computer, via further lines 38, which can also be bundled in a cable that extends from the glove device 10. In other embodiments, the component 36 comprises one or more pressure transducers that sense the pressure of the fluid within the force sensors 34. Irrespective of the nature of the component 36, communication between the force sensors 34 and the component can be enabled by lines (not shown) that extend from each force sensor to the component.

The glove device 10 of the embodiment of FIGS. 1 and 2 further comprises a motion sensor 40 that detects and measures motion of the device and the examiner's hand. In some embodiments, the motion sensor 38 comprises a piezoelectric, gyroscopic, or micro-electrical mechanical (MEMS) accelerometer. In such a case, acceleration of the glove device 10 is measured and that acceleration can be integrated over time to determine instantaneous velocity of the device and the examiner's hand.

The glove device 10 can be used to quantify weakness or changes in muscle tone such as spasticity or dystonia. In addition, the device 10 can measure acceleration of the body parts being tested while simultaneously measuring the forces required to move the body part. The changes in muscle tone at different velocities can help distinguish spasticity from dystonia, which is important in selecting appropriate therapies. Unlike machines that measure force and velocity, the device 10 instruments the examiner rather than the patient. This enables the examiner greater flexibility such that the device 10 can be used to assess substantially any joint that is clinically assessed. Moreover, the device 10 can be used to assess other parts of the body. For example, the device 10 can be used to palpate the abdomen.

The device 10 can be used to measure static forces, such as when measuring the patient's strength, or to measure muscle tone as the body part is moved. In some embodiments, some or all of the measurements collected by the device 10 are transmitted to a computer that receives the measurements and performs diagnostic analysis on the measurements. The measurements can be transmitted over the above-mentioned cable. Alternatively, the measurements can be wirelessly transmitted if the device 10 is provided with a wireless transmitter (not shown).

As discussed above, joint flexion causes lengthening of each strand 24. The joint flexions measured by each strand 24 can be used to determine the joint angle of each joint. In determining the joint angles, the contribution of more proximal joints on the lengthening of a strand can be subtracted to identify the particular rotation of a more distal joint. By calculating the joint angles, the relative angular orientation of the force sensors 34 of the finger sleeves can be determined. A force vector can then be calculated for each force sensor 34 and a resultant force vector can be calculated from the individual force vectors. Such a method of determining the resultant vector enables a large number of different examiner hand positions to accommodate examination of virtually any body part. Whether the examiner's hand is narrowly or widely closed, the grasp of the body part typically involves forces at several of the force sensors 34 and consequently, there will typically be a resultant vector that describes the balance of forces necessary to grasp and apply forces to the body part.

FIG. 3 illustrates this functionality. As is shown in that figure, an examiner wearing the glove device 10 has gripped an object 42 (e.g., a patient's wrist) and therefore has applied forces to the object 42 with the examiner's fingers, thumb, and palm. The force sensors 34 associated with those parts of the examiner's hand are therefore pressed against the object 42 and force measurements are obtained. Because the orientation (joint angles) of the fingers and thumbs are known from measurements made by the finger orientation sensors, the various force vectors (see arrows) associated with each force sensor 34 touching the object 42 can be determined. A resultant force vector can then be determined. At rest, the resultant force vector accounts for any static loads, such as gravity, to correct the subsequent forces determined during the assessment. During the active assessment of static muscle strength, the patient can be instructed not to let the examiner move the body part by applying muscular resistance. A new set of forces can then be determined and a new resultant vector can be calculated. The resultant vector calculated at rest can then be subtracted from the active assessment phase resultant vector to determine the actual resisting force applied by the subject. The same measurements can be obtained during passive movement of the body part.

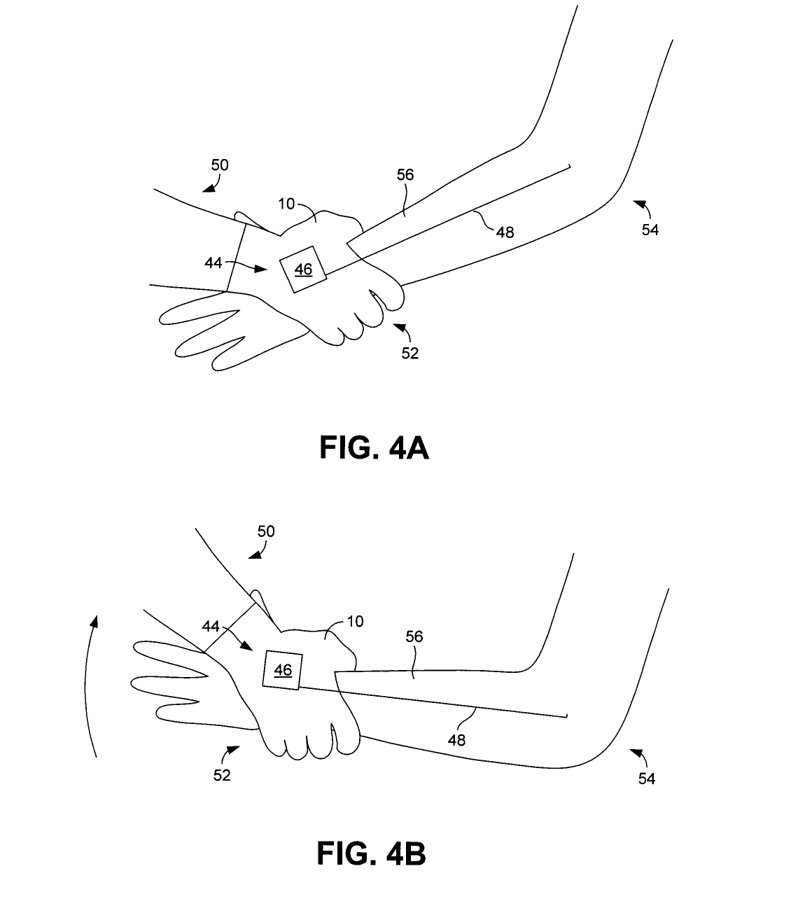

Although it is desirable to determine the resultant force vector, it is also desirable to determine the moment (torque) associated with the applied force. The moment can be determined with knowledge of the distance between the point at which the force is applied and the axis of rotation of the body part. In some embodiments, the glove device 10 further includes a distance measurement device for determining that distance. FIGS. 4A and 4B illustrate an embodiment of a glove device 10′ that includes a distance measurement device 44 in the form of a tape measure attached to the device having a body 46 and an extendible tape 48. FIGS. 4A and 4B illustrate use of the distance measurement device 44. As shown in the example of those figures, an examiner 50 can grip a patient's wrist 52 while wearing the glove device 10′. The distance measurement device 44 can be used to measure the distance between the examiner's hand and the patient's elbow joint 54 about which the examiner pivots the patient's forearm 56 (see FIG. 4B).

As is apparent from the above discussion, the disclosed glove device 10 is useful for the quantification of forces during the evaluation of muscle strength and/or tone. By instrumenting the examiner, the device 10 is highly adaptable to measure a wide range of forces in a variety of patients and conditions. The device 10 is also well suited for measuring the time course of resistance to passive movement, which is important in the evaluation of a number of neurological conditions such as dystonia and spasticity. Indeed, an important use of the device is in the evaluation of patients, particularly children, with spasticity, dystonia, and mixed spasticity/dystonia. The ability to measure spasticity and dystonia, and to distinguish spasticity from dystonia, is important to guiding therapy, such as dorsal rhizotomies, selective neurectomies, and intra-thecal and intraventricular medications such as baclofen.

The clinical condition of spasticity/dystonia in children with cerebral palsy is a clear illustration of the need to differentiate and quantify spasticity and dystonia. These children often are considered for invasive dorsal rhizotomy surgeries. While effective for spasticity, this surgery is ineffective for dystonia. Indeed, one of the leading causes of failure to relieve hypertonus with dorsal rhizotomy is the underestimation of the degree of dystonia involved. Some physicians, unsure of the relative degrees of spasticity and dystonia, will perform an intra-thecal test injection of baclofen at considerable expense and significant risk. The presumption (unproven) that the hypertonus that does not resolve indicates the degree of dystonia. Clearly, a simple, relatively inexpensive alternative such as the disclosed glove device 10 is needed. It may be possible to differentiate dystonia from spasticity by careful analysis of the change in resistance with the velocity of muscle stretch (joint rotation).

While particular embodiments have been discussed, many variations are possible. For example, in some embodiments, the glove device need not couple to a separate device during use. In such a case, the device can include its own power source, such as a battery.

1. A patient evaluation apparatus comprising:
a glove body adapted to be worn on an examiner's hand;
finger orientation sensors mounted to the glove body adapted to sense the orientation of the fingers and thumb of the examiner's hand;
force sensors mounted to the glove body adapted to measure forces applied against the examiner's hand; and
a motion sensor mounted to the glove body adapted to detect motion of the examiner's hand.

2. The apparatus of claim 1, wherein the glove body is made of a fabric or mesh material.

3. The apparatus of claim 1, wherein the glove body comprises a central portion that fits around the examiner's hand and finger sleeves that fit around the fingers and thumb of the examiner's hand.

4. The apparatus of claim 3, wherein the finger orientation sensors comprise linear potentiometers and strands that extend from the potentiometers.

5. The apparatus of claim 4, wherein a proximal end of each strands is connected to a linear potentiometer and a distal end of each strand is attached to a finger sleeve.

6. The apparatus of claim 5, wherein the distal ends of some of the strands attach to the finger sleeves near a tip of the finger sleeve and the distal ends of other strands attach to the finger sleeve near positions that correspond to joints of the examiner's hand.

7. The apparatus of claim 3, wherein the finger orientation sensors comprise optical fibers that extend from the central portion of the glove body to the finger sleeves.

8. The apparatus of claim 7, wherein distal ends of some of the strands attach to the finger sleeves near a tip of the finger sleeve and the distal ends of other strands attach to the finger sleeve near positions that correspond to joints of the examiner's hand.

9. The apparatus of claim 3, wherein the force sensors are provided or within the finger sleeves and the central portion of the glove body.

10. The apparatus of claim 9, wherein the force sensors are positioned on or within the finger sleeves to correspond to pads of the examiner's fingers and to the palm of the examiner's hand.

11. The apparatus of claim 1, wherein the force sensors are electronic force sensors.

12. The apparatus of claim 1, wherein the force sensors are hydraulic or pneumatic pads.

13. The apparatus of claim 1, wherein the motion sensor comprises an accelerometer.

14. The apparatus of claim 1, further comprising a distance measurement device mounted to the glove body.

15. The apparatus of claim 14, wherein the distance measurement device comprises a tape measure.

16. A glove device for evaluating muscle strength or muscle tone of a patient, the device comprising:
a glove body adapted to be worn on an examiner's hand, the body including a cuff that wraps around the wrist, a central portion that wraps around a central portion of the hand, and finger sleeves that wrap around the fingers and thumb;
finger orientation sensors mounted to the glove body adapted to sense the orientation of the fingers and thumb of the examiner's hand, the orientation sensors including strands that extend from the central portion of the glove body and attach to the tips of the finger sleeves or positions along the finger sleeve that correspond to joints of the examiner's hand;
force sensors mounted to the glove body adapted to measure forces applied against the examiner's hand, the force sensors being positioned on or within the finger sleeves at positions that correspond to pads of the examiner's fingers and thumb and to the examiner's palm;
an accelerometer mounted to the glove body adapted to detect motion of the examiner's hand; and
a distance measurement device mounted to the glove body.

17. The glove device of claim 16, wherein the finger orientation sensors further comprise linear potentiometers that are connected to the strands.

18. The glove device of claim 16, wherein the strands of the finger orientation sensors comprise optical fibers.

19. A method for evaluating muscle strength of a patient, the method comprising:
an examiner donning a glove device on the examiner's hand, the glove device including
the examiner grasping a body part of the patient with the hand on which the glove device is worn;
the examiner holding the body part steady while the patient attempts to move the body part;
measuring the forces applied to the glove device by the body part with sensors of the glove device;
determining the joint angles of joints of the examiner's hand with other sensors of the glove device; and
determining a force vector that identifies the magnitude and direction of a net force applied to the glove device.

20. A method for evaluating muscle tone of a patient, the method comprising:
an examiner donning a glove device on the examiner's hand, the glove device including
the examiner grasping a body part of the patient with the hand on which the glove device is worn;
the examiner moving the body part with the body part relaxed;
measuring the forces applied to the glove device by the body part with sensors of the glove device;
determining the joint angles of joints of the examiner's hand with other sensors of the glove device;
measuring a velocity of movement of the glove device with a further sensor of the glove device; and
determining a force vector that identifies the magnitude and direction of a net force applied to the glove device.
